(19) United States
(12) Patent Application Publication
(10) Pub. No.: US 2018/0249849 A1
(43) Pub. Date: Sep. 6, 2018

(54) COOLING BEACH BLANKET

(71) Applicant: Christopher Wilkinson, Brooklyn, NY (US)

(72) Inventor: Christopher Wilkinson, Brooklyn, NY (US)

(21) Appl. No.: 15/908,989

(22) Filed: Mar. 1, 2018

Related U.S. Application Data
(60) Provisional application No. 62/466,018, filed on Mar. 2, 2017.

(51) Int. Cl. A47G 9/02; A61F 7/00; A61F 7/10

(52) U.S. Cl. CPC A47G 9/0215; A61F 7/0097; A61F 2007/0219; A61F 2007/0056; A61F 2007/0094; A61F 7/106

(57) ABSTRACT

A personal cooling system for controlling personal body temperature at the beach comprises a first layer having a first edge region defining a size and a shape of the first layer, the first layer comprising a cooling gel pack, wherein the gel pack has an outer surface and a plurality of compartments for storing a temperature-controlling ingredient therein; a second layer having a second edge region defining a size and a shape of the second layer; and an outside bag, to an inside surface of the outside bag the first layer and the second layer are attached. The second layer in the system further comprises a liquid dispenser that is embedded in the mesh material of the second layer. The actions of the cooling system and the dispenser can be controlled by a remote controller.

### CLAIM OF PRIORITY

This application claims the priority of U.S. Ser. No. 62/466,018 filed on Mar. 2, 2017, the contents of which are fully incorporated herein by reference.

### FIELD OF THE EMBODIMENTS

This invention relates to a beach blanket as a personal cooling system and in particular to a beach blanket with several storage compartments for holding, among other things, a cooling pack and a dispenser for sun screen, and is easy to carry.

### BACKGROUND OF THE EMBODIMENTS

Each year in the summer season, people go to the beach on the sea/lake shores to enjoy various activities in the hot summer air. These activities can include an occasional dipping or swimming in the cool water, as well as more time spent as a lazy afternoon sunbathing in the warm summer sun. While the health and psychological benefits afforded by such recreational activities are many and well recognized, there remains an unmet need for providing beachgoers with a beach blanket equipped with means to protect themselves from potential harms brought by the scorching heat and UV light under the sun, especially for the cooling needs for those beachgoers who prefer activities other than dipping in the water.

Typically for beach-going, people need to carry with them many different articles for a variety of purposes: swimming, playing, reading, entertainment, eating and drinking, and personal hygiene and protection, etc. In addition, the beachgoers may also need to carry other larger beach equipment, such as beach chairs and coolers. As a result, it is clearly highly desirable to have products for beachgoer-use that can serve a multiple of the aforementioned purposes. More desirable for those beachgoers who prefer activities other than dipping in the water is a personal cooling system for body temperature control that can also function as serve as a reservoir for the skin care and other needs, such as sun-screens for those beachgoers who prefer to stay dry under the sun.

Examples of related art are described below: One of the potential solutions for the aforementioned problems is disclosed in U.S. Pat. No. 5,346,308 granted to Buhot et al., which is related to a bag with storage pocket which converts into a beach towel comprising a bag front wall attached to a beach towel and a flap. The flap further comprises a zippered storage compartment. One or more shoulder straps are attached to the bag with storage pocket which converts into a beach towel in order to provide hands-free transportation of the items contained in the bag with storage pocket which converts into a beach towel and the storage compartment. However, the bag does not contain any cooling mechanisms.

U.S. Pat. No. 5,005,374 to Spitler relates to a thermal wrap includes a fabric band having a thermally insulating outer layer and a non-insulating inner layer. A cavity is formed between the two layers for receiving a flexible cold pack containing deionized water and a chemical freezing agent. In one embodiment, the fabric band is in the form of a collar to be worn about the individual's neck or other body part. In another embodiment, the fabric band is in the form of a jacket for surrounding a beverage container such as a wine bottle. But this is not suitable to be used as a beach blanket.

U.S. Pat. No. 6,257,011 to Siman-Tov et al. relates to a portable lightweight personal cooling apparatus with a channeled sheet which absorbs sweat and/or evaporative liquid, a layer of highly conductive fibers adjacent the channeled sheet; and, an air-moving device for moving air through the channeled sheet, wherein the layer of fibers redistributes heat uniformly across the object being cooled, while the air moving within the channeled sheet evaporates sweat and/or other evaporative liquid, absorbs evaporated moisture and the uniformly distributed heat generated by the human body, and discharges them into the environment. This not only is not a beach blanket, but actually does not generate any below-ambient temperatures felt by the user.

U.S. Pat. No. 5,391,198 to Cheney et al. relates to a method of applying heat or cold to human or animal parts and a reusable compress utilized therein. The method involves saturating crystals of a water soluble acrylic polymer and a thickening agent through the porous fabric of a compress bag containing the acrylic polymer and the thickener. The compress is heated in a warm water or a microwave oven or cooled in a cool environment. The compress is applied to a human or animal part to warm or cool the body part. When the compress is no longer needed the gel is de-hydrated and the compress is easily stored for safe reuse.

U.S. Patent Application No. 2008/0039913A1 discloses an aromatherapy blanket includes a flexible, aroma-permeable cover that has upper and lower layers. The upper and lower layers are joined at their outer edges and define a space between them. The space is divided into a number of subspaces which are filled with a quantity of thermal reservoir material and a quantity of aromatherapy material. The typically tube-shaped subspaces are spaced apart from one another by a first predetermined distance. The subspaces are divided into substantially equal portions to prevent shifting of the thermal material and the aromatherapy material. The thermal reservoir materials are selected from the group consisting of corn, rice, wheat, oats, barley, beans and flaxseed. The aromatherapy materials are selected from the group consisting of cinnamon, chamomile, lemongrass, peppermint, rosemary, spearmint, valerian root, white willow, yarrow, lavender ginger and yellow dock root. The blanket includes a washable containment cover sized and shaped to enclose the blanket.

U.S. Pat. No. 6,132,455 issued to Shang relates to a thin layer of a cooling composition having a novel and narrow relative range of components, where such thin layer is maintained as such in a thin sheath for use as a cooling cushion especially for the seat of a sitting human, but may also be used for the back of a reclining human. The use of this cushion is for ambient temperatures at about 75.3° F. (23° C.) and above, i.e., the initial melting temperature for the cooling composition incorporating a specific phase change material.

Chinese utility model patent No. 201282760Y issued to Wang relates to a ventilation sunscreen towel and cap, comprising a towel, a cap body, a hidden air opening and a connecting belt, which is characterized in that the towel and the cap bodies are provided with a hidden air opening formed by an air opening and an upper latch sheet, or an upper latch sheet and a lower latch sheet; the edge of the upper latch sheet of the hidden air opening is embedded or packed with rigid material or is coiled with cloth edge, to open the hidden air opening in arc form. The ventilation sunscreen towel and cap has reasonable design, easy manufacture, sunscreen and ventilation property, good effect, and simple application.

Chinese utility model patent No. 2375127Y issued to Lu et al. relates to a multi-purpose towel protecting against sunshine. A shoulder cape has a lacing, and a shoulder protector or a protecting sleeve is arranged between the shoulder cape and the lacing. The utility model has the advantages of reasonable structure, good material quality, a multi-purpose towel, convenient operation, low cost, etc., and water breaks away from the utility model. The utility model solves the problems that skin is easily damaged by sunshine and a bath towel is not easily dewatered and carried, etc., when people swim out of room. The utility model particularly adopts special materials, dewatering speed is quick, and cost is low. The utility model can be used as a dress smock for being sun-proof and can be used as a kerchief and a towel, and the utility model combines the functions of beautiful, practical and sun-proof and realizes the effect of the multi-purpose towel. So the utility model can serve an ideal swimming necessary article.

None of the related art described above addresses all of the issues that the present invention does. The various embodiments of the present invention can provide beachgoers with a multi-functional beach blanket that is easy to carry and has a built-in personal cooling system for body temperature control and a reservoir for storing/dispensing sun-screens, both of which can be controlled and regulated through a remote control, and is especially suited to those beachgoers who prefer not going into water, but staying dry under the hot sun.

### SUMMARY OF THE EMBODIMENTS

In one of the embodiments, the present invention provides a system for controlling personal body temperature that comprises a first layer having a first edge region defining a size and a shape of the first layer, the first layer comprising a gel pack, wherein the gel pack has an outer surface and a plurality of compartments storing a temperature-controlling gel therein; a second layer having a second edge region defining a size and a shape of the second layer; and an outside bag, to an inside surface of which the first layer and the second layer are attached.

In accordance with one aspect of the present invention, the gel pack in the aforementioned system defines the first edge region of the first layer, which further comprises a first material that covers the outer surface of the gel pack.

In accordance with another aspect of the present invention, the aforementioned first material includes an opening having a size and shape that are substantially the same as the size and shape of the gel pack, wherein the gel pack is positioned in said opening of the first material and securely fastened to said first material by stitching, adhesive or hook and loop fasteners about a periphery of the gel pack.

In accordance with yet another aspect of the present invention, the aforementioned system further comprises a skirt disposed along an outer periphery of the outside bag, and in addition, a closure fastener disposed along a periphery of the outside bag and a mating closure fastener disposed along another periphery of the outside bag, such that the outside bag may be selectively opened or closed with the closure fastener and the mating closure fastener.

In another one of the embodiments, the present invention provides that the gel pack in the aforementioned system is made from a polymer material and filled in its multi-compartments with i) a cooling composition that is capable of generating a below-ambient temperature for a period of time; and ii) a phase change material that prevents extreme cold feel and moderates the minimum cold temperature generated, thus moderating the cooling effect and prolonging the period of time during which the cold temperature is generated.

In accordance with yet another aspect of the present invention, the aforementioned cooling composition can be selected from a wide variety of suitable compositions and is preferably selected from the group consisting of ice cubes, dry ice, heat-storing antifreeze solution, polymeric cold-retaining agents, and endothermic ingredients, wherein the cooling composition is packed in a water-impermeable bag or other such receptacle. Here, the endothermic ingredient, upon being dissolved in water (that is, optionally, supplied from a separate package and introduced via, for instance, a syringe pump), is capable of slowly absorbing heat forming an endothermic reaction, wherein the amount of water and the speed of its introduction into the endothermic ingredient can relate to the cold temperature thus generated, which temperature is therefore capable of being regulated by controlling the power supplied to the syringe pump. The controlling of the power can, in turn, be accomplished via a wired, or wireless, mechanism.

In accordance with still another aspect of the present invention, a wide variety of suitable materials can serve as the aforementioned phase change material, which undergoes a phase transition at a temperature of from about 3° C. to about 25° C., thus suitably providing a cool but comfortable temperature to the user, while avoiding the user to be exposed to extremely cold temperatures. Preferably, the phase change material is in the form of fluid or gel and is selected from the group consisting of liquid, linear or branched hydrocarbon oils, liquid fatty alcohols or derivatives, and mixtures thereof.

In accordance with still another aspect of the present invention, the first material in the aforementioned system is made from a flexible foil material that is selected from the group consisting of plastic, thermoplastic and rubber based materials, while the second layer is made from a fabric or mesh woven material that is selected from the group consisting of cotton cloth, linen, wool, and synthetic fabrics.

In accordance with yet another aspect of the present invention, the second layer in the aforementioned system further comprises a liquid dispenser that is embedded in the mesh material of the second layer and this liquid stored in the liquid dispenser is selected from the group consisting of sunscreen, body lotion, moisturizer, and insect repellent sprays.

In accordance with yet another aspect of the present invention, the outside bag in the aforementioned system is made from a terri-cloth or fabric material.

In another one of the embodiments, the present invention provides that the aforementioned personal cooling system further comprises two individual thermally insulated panels on two sides of the bag, wherein there are built-in fans and ventilation disposed on the panels to allow the cooled air from the cooling gel pack to travel through the side panels and filter through the vents, thus allowing the cooled air to circulate throughout the entire towel bag.

In still another one of the embodiments, the present invention provides that a water reservoir compartment is disposed to an inside part of the aforementioned system and is connected through a tubing to an inlet of a water pump, the outlet of which is connected through another tubing to the gel pack, wherein the cooling composition comprise a chemical, when is dissolved in water, can cause an endothermic reaction to occur, which chemical is selected from the group consisting of endothermic ingredients. As such, the amount of water introduced, through said pump, into the gel pack is proportionally related to the extent of the endothermic reaction and therefore, the extent of the resulted cooling of the gel pack, which extent is further adjustable and maintained by the presence, in a separate compartment of the gel pack, of a phase change agent, which is selected from the group consisting of liquid, linear or branched hydrocarbon oils, liquid fatty alcohols or derivatives, and mixtures thereof.

In accordance with another aspect of the present invention, the aforementioned water pump can effectuate a slow, but steady, introduction of water into said chemical in the gel pack, which, when dissolved in water, is capable of causing an endothermic reaction. When coupled with an appropriate phase change agent present in the vicinity, this endothermic reaction can lead to an overall lasting effect of the cooling felt by the user. The extent of the cooling (i.e., the cold temperature of the gel pack) and its rate of change are both determined by the actions of the water pump, which action is powered and controlled through electronic circuitries that are disposed on an inside surface of the bag. The electronic circuitries are, in turn, powered by batteries, or solar panels, disposed on an outside surface of the bag and controlled by a control panel with an LCD screen and push buttons also disposed on another outside section of the bag.

In accordance with yet another aspect of the present invention, the present invention provides that a sunscreen/liquid reservoir is disposed to an inside part of the aforementioned system and is connected through a tubing to an inlet of a liquid pump, the outlet of which is connected through another tubing to the dispenser, wherein the aforementioned liquid pump can effectuate a slow, but steady, flow of a liquid, such as UV sunscreen into the dispenser. The amount of UV sunscreen supplied to the dispenser is determined by the action of the liquid pump, which action is powered and controlled through electronic circuitries that are disposed on an inside section of the bag. The electronic circuitries are, in turn, powered by regular batteries, or solar panels, disposed on an outside surface of the bag and controlled by a control panel with an LCD screen and push buttons also disposed on another outside surface of the bag.

In accordance with still another aspect of the present invention, the liquid/UV sunscreen stored in aforementioned reservoir and dispensed from said dispenser is selected from the group consisting of sunscreen, body lotion, moisturizer, and insect repellent sprays.

In still another one of the embodiments, the present invention provides that the temperature controlling action of the gel pack and the action of the UV sunscreen/liquid dispenser are remotely controllable by a remote controller with push buttons. The controlling actions of this remote controller are effectuated through the aforementioned electronic circuitries by way of WIFI and/or Bluetooth.

In accordance with yet another aspect of the present invention, the remote controller is powered by regular batteries or solar panels that are mounted on an outside surface of the controller.

It is an object of the present invention to provide a multi-functional personal cooling system for controlling the body temperature of an user in the form of a beach blanket, which also contains a means to store and dispense skin care/protection liquids, such as sun-screens, both the cooling and the dispensing can be controlled and regulated through a control panel embedded in the blanket and further controllable by a remote controller.

### BRIEF DESCRIPTION OF THE DRAWINGS

FIG. 1 shows a top view of one of the embodiments of the invention in an open configuration.

FIG. 2. shows a top view of the same embodiment as in FIG. 1, but in a zip-closed configuration.

FIG. 3. shows a top view of another embodiment of the invention (in a zip-closed configuration).

FIG. 4. shows a top view of another embodiment (remote control) of the invention

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

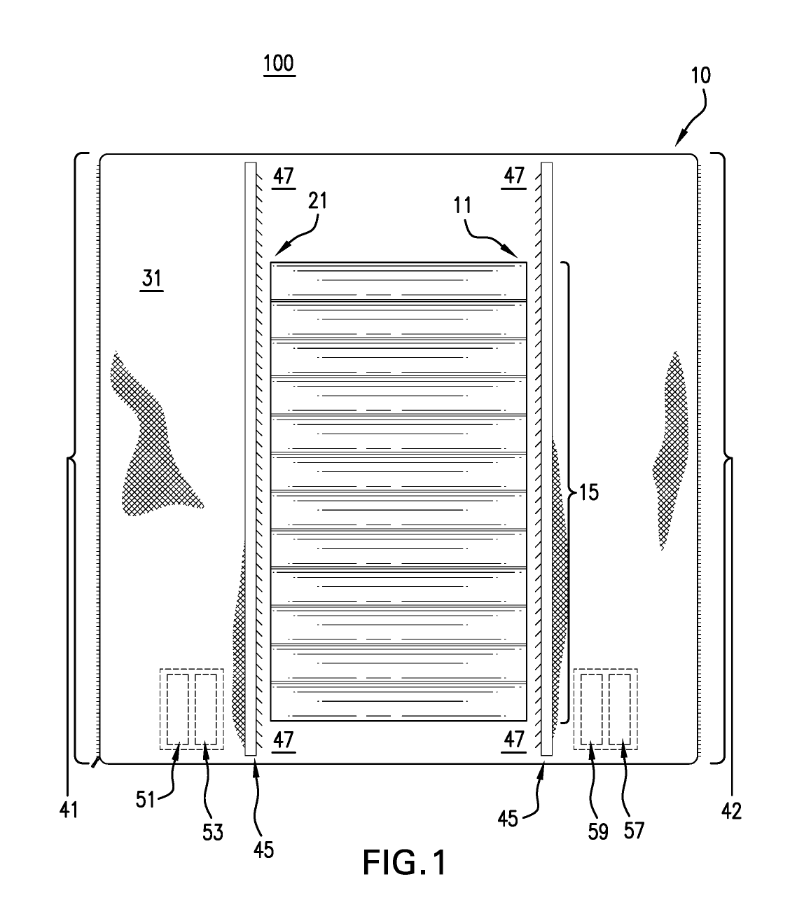

Referring now to the drawings, and to FIG. 1 in particular, which is, according to an embodiment of the invention, a top view of a personal cooling system 100, which consists of a beach blanket 10 with a cooling gel pack 15. The personal cooling system 100 may include a first layer 11 having a first edge region defining a size and a shape of the first layer 11, the first layer further includes a topical cooling insert/gel pack 15, wherein the gel pack 15 has an outer surface and a plurality of compartments used for storing a temperature-controlling ingredient therein; a second layer 21 having a second edge region defining a size and a shape of the second layer 21; and an outside bag 31, to an inside surface of which the first layer 11 and the second layer 21 are attached.

The gel pack 15 in the aforementioned personal cooling system 100 defines the first edge region of the first layer 11, which further comprises a first material that covers the outer surface of the gel pack 15 and the first material includes an opening having a size and shape that are substantially the same as the size and shape of the gel pack 15, wherein the gel pack 15 is positioned in said opening of the first material and securely fastened to said first material by stitching, adhesive or hook and loop fasteners about a periphery of the gel pack 15.

In accordance with still another aspect of the present invention, the first material in the aforementioned personal cooling system 100 is made from a foil/polymer material that is selected from the group consisting of plastic, thermoplastic and rubber based materials, while the second layer 21 is made from a fabric or mesh material that is selected from the group consisting of cotton cloth, linen, wool, and synthetic fabrics. Examples of the first material can include vinyl, urethane, copolymer latex of butadiene-acrylonitrile, copolymer of vinylidene chloride-acrylic, resinous latex, rubber latexes, epoxy polymers, polyurethane polymers, acrylic polymers, cellulose acetate, polyamides, any resin, or the like.

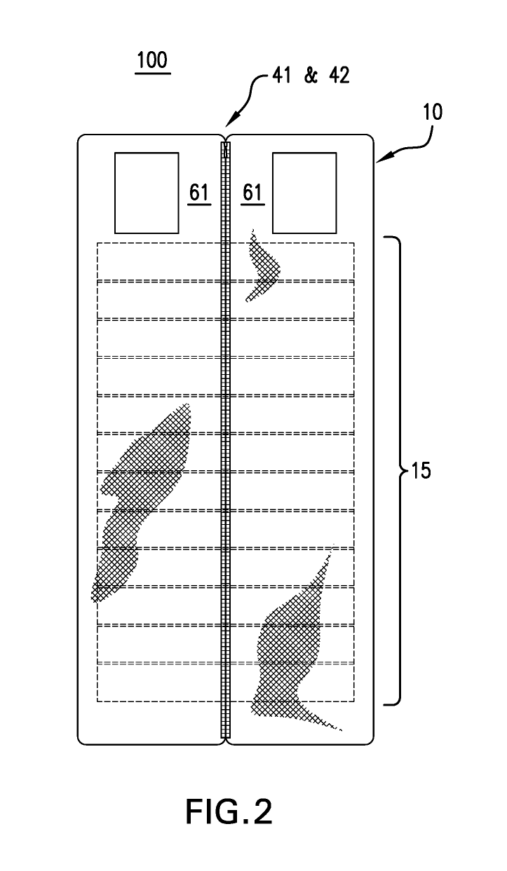

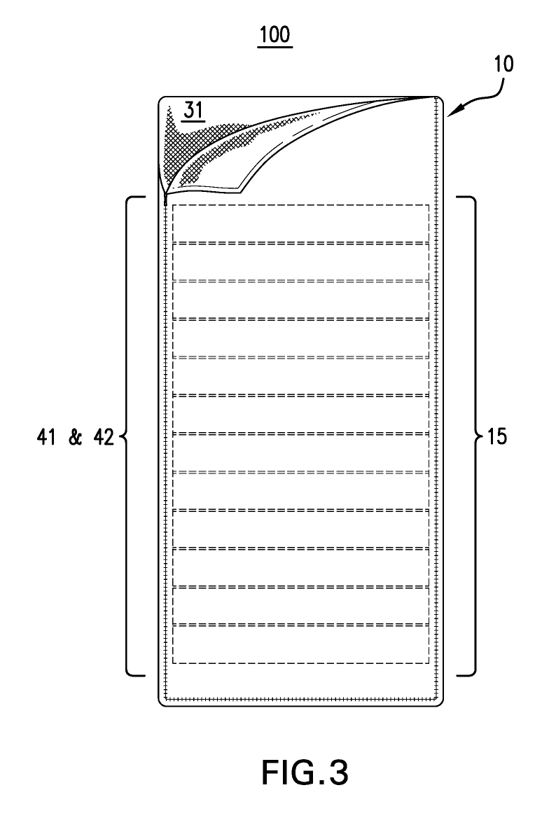

Referring now to FIGS. 1, 2 and 3, the personal cooling system 100 further comprises a skirt disposed along an outer periphery of the outside bag, and in addition, a closure fastener 41 disposed along a periphery of the outside bag and a mating closure fastener 42 disposed along another periphery of the outside bag 31, such that the outside bag 31 may be selectively opened or closed with the closure fastener 41 and the mating closure fastener 42. Here, FIG. 1 shows a top view of one of the embodiments of the invention in an open configuration, while FIG. 2 shows the same embodiment in a closed configuration. Analogously, FIG. 3 shows the same embodiment in a closed configuration. The outside bag 31 in the aforementioned system 100 is made from a terri-cloth or fabric material.

According to another one of the embodiments, the present invention further provides that the personal cooling system 100 further comprises two individual thermally insulated panels 45 on two sides of the bag 31, wherein there are built-in fans and ventilation disposed on the panels 45 to allow the cooled air from the cooling gel pack 15 to travel through the side panels 45 and filter through the vents 47, thus allowing the cooled air to circulate throughout the entire towel bag 31.

While the personal cooling system 100 of the present invention preferably has a suitable flexibility such that it can be applied to a human body, and/or equally suitable, to the body of an animal body. Herein, the personal cooling system 100 of the present invention is described primarily in terms of human use, but it should be understood that the cooling effects and benefits herein can also be used with equal advantage for animals. As a result, it should be understood that the personal cooling system 100 of the present invention is not limited to human use.

According to another one of the embodiments, the present invention provides that the gel pack 15 in the aforementioned personal cooling system 100 is made from a foil/polymer material and filled with an ingredient consisting of a cooling composition that is capable of generating, for a period of time, a range of below-ambient temperatures therefore sensible by a user, as well as a phase change material that prevents an extreme cold feel and moderates the minimum cold temperature thus generated by the cooling composition, and therefore moderates the cooling effect it supplies and prolongs the period of time during which the below-ambient temperature is felt by the user.

According to another one of the embodiments, the present invention further provides that the gel pack 15 is made from at least one flexible polymer material, which means a material that allows the user to comfortably fit it to the user's body. Preferably, the flexible polymer materials herein are made of any polymer materials to impart a soft feel, which can include, for example, an elastic film which is typically a synthetic resin film having sufficient strength during use and transport, and the like. Examples of the synthetic films include polyethylene, polypropylene, polyester, nylon, polyvinyl chloride, polyvinylidene chloride, polyurethane, and other synthetic resins. Composite and laminate films of these materials are also available. Polyester, nylon, polyethylene, polypropylene, and composite films, blends, or laminates of these materials are preferred.

According to another one of the embodiments, the phase change material of the present invention adjusts the minimum temperature felt by the user of the aforementioned personal cooling system 100. While not wishing to be bound by theory, the cold temperature generated from the cooling composition in the gel pack 15, which is generally from about 5 to about 20° C., is believed to allow the phase change material to undergo a transition from liquid to solid and such external energy absorption by the phase-change material is believed to control the cold temperature to levels appropriate for the user body. The absorbed cold temperature is further believed to be released from the phase change material to be felt by the user and due to the characteristic of the phase change material, the releasing speed for the temperature from the phase change material tends to be slower than when the gel pack 15 containing only the cooling composition contacts the user body directly. The presence of the phase change material also physically prevents extreme chill feeling. Therefore, the phase change material is preferably positioned between the user body and the cooling composition.

The personal cooling system 100 of the present invention provides the user with a pleasant feeling of a below-ambient temperature without extreme cold and for an extended period of time. Preferably, the below-ambient temperature generated from the personal cooling system 100 is less than about 30° C. In addition, the below-ambient temperature thus generated may be maintained for a period of time that can depend on various factors, e.g., ranging from periods of about 20 minutes to periods of about 4 hours after the below-ambient temperature is first generated.

While many suitable cooling compositions are known and may be used in the aforementioned personal cooling system 100, it is very desirable, in accordance with another aspect of the present invention, that such a cooling composition is non-toxic and safe to humans and does not have an offensive odor. Preferably, such cooling compositions are selected from the group consisting of dry ice, water-based compositions such as ice cubes, cold-water, heat-storing antifreeze solution, polymeric cold-retaining agents, and endothermic ingredients.

While dry ice is for one-time use only, various water-based cooling compositions are, of course, re-useable, by way of, for example, being refrigerated after the previous use. On the other hand, the endothermic ingredient, upon combining with water, generates an endothermic reaction. A variety of endothermic ingredients are useful herein provided that the ingredient(s) are capable of slowly absorbing heat when dissolved in water. The endothermic ingredients useful herein include inorganic salts, alkali metal salts, calcium salts, ammonium salts, urea, simple saccharides, and mixtures thereof. Sodium phosphate salts, sodium ammonium phosphate salts, and ammonium halide and nitrate salts are generally preferred due to their relatively high heat absorption and ability to maintain fine crystal morphology despite repeated dissolving and re-precipitation of crystals. More preferred are ammonium halides and ammonium nitrate.

Examples of sodium phosphate salts are disodium hydrogen phosphate, sodium dihydrogen phosphate, trisodium phosphate, and hydrates thereof. Examples of sodium ammonium phosphate salts and ammonium phosphate salts are sodium ammonium hydrogen phosphate, diammonium hydrogen phosphate, ammonium dihydrogen phosphate, triammonium phosphate, and hydrates thereof. These salts may be used singly or in combinations of two or more; preferably, the combination of at least one sodium phosphate salt and at least one sodium ammonium phosphate salt or ammonium phosphate salt. Examples of inorganic salts other than phosphates are sodium carbonate, sodium hydrogen carbonate, potassium sodium carbonate, sodium chromium carbonate, sodium scandium carbonate, sodium cerium carbonate, sodium sulfate, and hydrates of the above. These may be used alone, but preferably are used with the phosphates or other inorganic salts listed above for the purpose of controlling crystal precipitation and dissolving temperature.

While many suitable phase change materials are known and may be used in the aforementioned personal cooling system 100, it is also very desirable, in accordance with yet another aspect of the present invention, that such a phase change material is non-toxic and safe to humans and does not have an offensive odor. Preferably, such phase change materials are selected from the group consisting of liquid, linear or branched hydrocarbon oils, liquid fatty alcohols or derivatives, and mixtures thereof. Examples of phase changing materials include pivaldehyde, 3-bromoacetophenone, acrylic acid, adiponitrile, 2-nitroanisole, 3-chlorobenzaldehyde, benzene, ortho-dichlorobenzene, 3,3′-dimethylbiphenyl, 3,3-dimethyl-2-butanol, 1-hexadecene para-cresol, cylcooctane, 1-decanol, diethylsulfoxide, 1,4-dioxane, ethanolamine, formic acid, 1-heptadecene, 1-hexadecyne, methyl laurate, nonanoic acid, olelyl alcohol, 1-pentadecene, methacrylic acid, pyruvic acid, methyl sorbate, tetradecane, pentadecane, among others.

Now referring to FIGS. 1 and 2 in still another one of the embodiments, the present invention provides that a water reservoir 51 is disposed to an inside part of the aforementioned personal cooling system 100. The reservoir 51 is connected through a tubing to an inlet of a water pump 53, while the outlet of the water pump 53 is connected through another tubing to the cooling gel pack 15, wherein the cooling composition comprise a type of chemicals, as mentioned above, can cause an endothermic reaction to occur when is dissolved in water. This type of chemicals is selected from the group consisting of polymeric cold-retaining agents, and endothermic ingredients. As such, the amount of water introduced, through the water pump 53, into the gel pack 15 is proportionally related to the extent of the endothermic reaction occurred, which, in turn, determines the extent of the resulted cooling of the gel pack 15. The extent of the cooling is further adjustable and maintained by the presence of an appropriate phase change agent in a separate compartment of the gel pack 15. This phase change agent is selected from the group consisting of liquid, linear or branched hydrocarbon oils, liquid fatty alcohols or derivatives, and mixtures thereof.

According to such an embodiment, the aforementioned water pump 53 can effectuate a slow, but steady, introduction of water into the gel pack 15 containing said chemical, which is capable of causing an endothermic reaction when dissolved in water. When coupled with an appropriate phase change agent present in the vicinity of the chemical, the resulting endothermic reaction can lead to an overall lasting effect of the cooling felt by the user. The extent of the cooling, which is related to the resulting cold temperature of the gel pack 15 and the rate of the temperature change are both determined by the actions of the aforementioned water pump 53. The water pump 53 is powered and controlled through electronic circuitries that are disposed on an inside surface of the bag 31. The electronic circuitries are, in turn, powered by regular batteries, or solar panels, disposed on an outside surface of the bag 31 and controlled by a control panel 61 with an LCD screen and push buttons also disposed on another outside section of the bag 31 (FIG. 2).

According to still another embodiments, the present invention further provides that the second layer 21 in the aforementioned personal cooling system 100 comprises a liquid dispenser that is embedded in the mesh material of the second layer 21. In accordance with yet another aspect of the present invention, a sunscreen/liquid reservoir 57 is disposed to an inside part of the aforementioned system and is connected through a tubing to an inlet of a liquid pump 59. The outlet of the liquid pump is connected through another tubing to the aforementioned liquid dispenser. As such, the liquid pump 59 can effectuate a slow, but steady, flow of a liquid, such as UV sunscreen, into the liquid dispenser and the amount of UV sunscreen supplied to the dispenser is determined by the action of the liquid pump. This liquid pump 59 is powered and controlled through electronic circuitries that are disposed on an inside section of the bag 31. The electronic circuitries are, in turn, powered by regular batteries, or solar panels, disposed on an outside surface of the bag 31 and controlled by a control panel 61 with an LCD screen and push buttons also disposed on another outside surface of the bag 31 (FIG. 2).

In accordance with still another aspect of the present invention, the liquid/UV sunscreen stored in aforementioned reservoir 57 and dispensed from said dispenser is selected from the group consisting of sunscreen, body lotion, moisturizer, and insect repellent sprays.

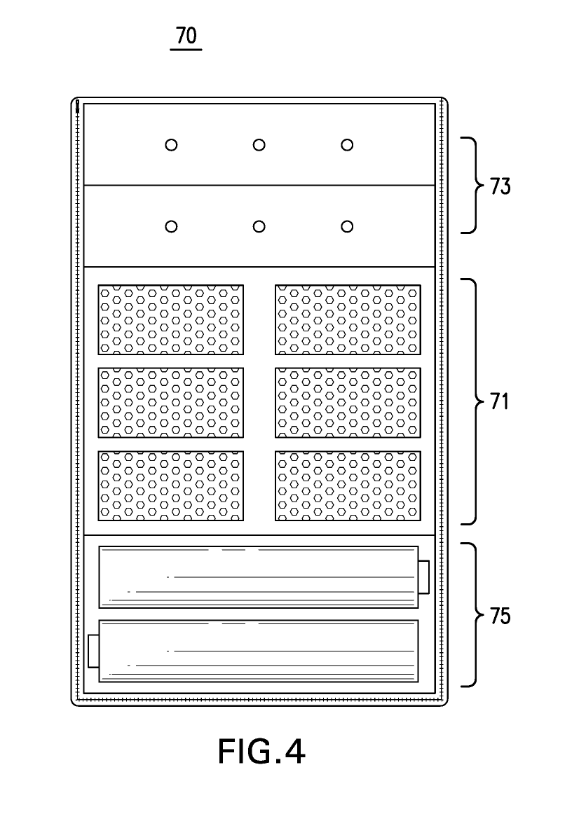

In still another one of the embodiments of the present invention, the controlling of the extent and rate of change of the temperature of the gel pack 15 and that of the action of the UV sunscreen/liquid dispenser are remotely controllable by a remote controller 70 with push buttons. Referring now to FIG. 4, which shows such a remote controller 70 for initiating and adjusting the temperature controlling action of the gel pack 15 and the action of the liquid dispenser embedded in the second material. The controlling actions of this remote controller 70 are effectuated through communication by way of the aforementioned electronic circuitries via WIFI and/or Bluetooth. The remote controller 70 is powered by regular batteries, or solar panels 71 that are mounted on an outside surface of a middle section of the remote controller 70, which are used in the place, or in conjunction, of the regular batteries 75 to power the controller 70. Disposed in a top section of the remote controller are two rows of push buttons 73, one row for controlling the cooling action of gel pack 15 and another for controlling the action of the liquid dispenser.

The various embodiments of the present invention therefore provide beachgoers with a multi-functional beach blanket that is easy to carry and has a built-in personal cooling system for body temperature control and a reservoir for storing/dispensing sun-screens, both of which can be controlled and regulated through a remote control, and is especially suited to those beachgoers who prefer not going into water, but staying dry under the hot sun.

The many elements of the present invention make it unique in the field. The novelty is illustrated by the various options for nearly every aspect of the invention that allow it to be used in the proper exercise form by a variety of users, both in terms of body size and fitness level. Additionally, there is a wide range of exercises available to any user of the present invention, and users can perform exercises that use the upper and lower extremity muscle groups simultaneously.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system for body temperature control comprising:
a first layer having a first edge region defining a size and a shape of the first layer, the first layer comprising a gel pack, wherein the gel pack has an outer surface and a plurality of compartments storing a temperature-controlling gel therein;
a second layer having a second edge region defining a size and a shape of the second layer; and
an outside bag, to an inside surface of which the first layer and the second layer are attached.

2. The system of claim 1, wherein the gel pack defines the first edge region of the first layer.

3. The system of claim 1, wherein the first layer further comprises a first material that covers the outer surface of the gel pack.

4. The system of claim 3, wherein the first material includes an opening having a size and shape, wherein the gel pack has a size and shape substantially the same as the size and shape of the opening, and the gel pack is positioned in and securely fastened to said opening of the first material by stitching, adhesive or hook-and-loop fasteners about a periphery of the gel pack.

5. The system of claim 1, further comprising a skirt disposed along an outer periphery of the outside bag, and in addition, a closure fastener disposed along a periphery of the outside bag and a mating closure fastener disposed along another periphery of the outside bag, such that the outside bag may be selectively opened or closed with the closure fastener and the mating closure fastener.

6. The system of claim 1, wherein the gel pack is made from polymer material.

7. The system of claim 1, further comprises two individual thermally insulated panels disposed on two sides of the outside bag, wherein there are built-in fans and ventilation disposed on the panels to allow the cooled air from the cooling gel pack to travel through said side panels and through the ventilation to circulate throughout the entire system.

8. The system of claim 1, wherein the gel pack is filled with i) a cooling composition that is capable of generating a below-ambient temperature for a period of time; and ii) a phase change material that prolongs the period of time during which the cold temperature is generated.

9. The system of claim 8, wherein the cooling composition is selected from the group consisting of dry ice, water-based compositions such as ice cubes, cold-water, heat-storing antifreeze solution, polymeric cold-retaining agents, and endothermic ingredients.

10. The system of claim 8, wherein the phase change material is selected from the group consisting of liquid, linear or branched hydrocarbon oils, liquid fatty alcohols or derivatives, and mixtures thereof.

11. The system of claim 1, wherein the first material is made from a foil material that is selected from the group consisting of plastic, thermoplastic and rubber based materials.

12. The system of claim 1, wherein the second layer is made from a fabric or mesh material that is selected from the group consisting of cotton cloth, linen, wool, and synthetic fabrics.

13. The system of claim 1, wherein the second layer further comprises a liquid dispenser that is embedded in the mesh material of the second layer.

14. The system of claim 13, wherein the liquid dispensed through the liquid dispenser is selected from the group consisting of sunscreen, body lotion, moisturizer, and insect repellent sprays.

15. The system of claim 1, wherein the outside bag is made from a terri-cloth or fabric material.

16. The system of claim 1, wherein the temperature controlling action of the gel pack and the action of the liquid dispenser are controlled by an on-board controller.

17. The system of claim 16, wherein the on-board controller is powered by regular batteries or solar panels that are mounted on an outside surface of the outside bag.

18. The system of claim 1, wherein the temperature controlling action of the gel pack and the action of the liquid dispenser are remotely controllable by a controller.

19. The system of claim 18, wherein the remote controller is powered by regular batteries or solar panels that are mounted on an outside surface of the controller.
